(19) United States
(12) Patent Application Publication
(10) Pub. No.: US 2017/0360542 A1
(43) Pub. Date: Dec. 21, 2017

(54) BANDAGE

(71) Applicant: Jeffery Thomas Brasch, Kelso (AU)

(72) Inventor: Jeffery Thomas Brasch, Kelso (AU)

(21) Appl. No.: 15/627,823

(22) Filed: Jun. 20, 2017

(30) Foreign Application Priority Data
Jun. 21, 2016 (AU) 2016902428
Oct. 14, 2016 (AU) 2016101806

(51) Int. Cl. A61D 9/00; A61L 15/28; A61F 13/00; A61F 13/02

(52) U.S. Cl. CPC A61D 9/00; A61F 13/0273; A61F 13/0206; A61L 15/28; A61F 13/0283; A61F 13/00063; A61F 13/0269; A61F 13/0266; A61F 13/0253; A61F 2013/00119; A61L 2300/412

(57) ABSTRACT

A bandage for the treatment of animal wounds and related method includes use of a flexible kinesiology tape, a pressure-sensitive adhesive and an absorbent pad disposed on an inner face of the kinesiology tape. The pressure-sensitive adhesive retains the absorbent pad on the tape while a residual portion is free to adhere in use to itself or a surface such as a skin surface surrounding the wound to retain the bandage in position.

### TECHNICAL FIELD

Disclosed herein is a bandage for the treatment of animal wounds. The bandage is disclosed in the context of use for the treatment of equine wounds. However, the bandage may be used for the treatment of animals (e.g. cattle, sheep, dogs, cats) and humans.

### BACKGROUND ART

Veterinarians and owners of animals bandage limbs and other parts of the animal to protect wounds and surgical sites. A bandage can prevent contamination, provide compression to minimize swelling and hold topical medications against the wound.

For equine limb wounds, a layered bandage is commonly applied. Once the wound is cleaned, a medicated dressing may be applied as a primary bandage to promote autolytic debridement. The medicated dressing may then be covered with a thick layer of padding and secured to the wound with a further layer of material that is wrapped around the equine limb.

Problems with traditional bandaging methods include that the wounded limb may be immobilised upon application of the bandage. Immobilisation of a limb may prevent blood flow in the region of the wound and inhibit healing. Further, traditional bandages can require multiple people to apply and, if not applied properly, cause wrinkles or bunches of padding in the region of the wound. This can produce pressure points that are uncomfortable for the animal.

Traditional bandages may also need to be monitored daily to ensure that the bandage is not tightening, loosening or slipping in the region of the wound. In addition to the material and application (e.g. if a veterinarian is required to apply the bandage) being expensive, bandages may need to be replaced often to ensure that the wound site does not become infected and to restore movement and circulation to the region of the wound. Replacement of the bandage can increase the cost of the healing process.

In the equine industry, traditional bandaging may also cause problems such as bandage burns and bandage bows from pinched tendons. Further, it may be difficult to apply a bandage to a horse without allowing dust and dirt to slip down the top of the bandage and enter the wound.

It is to be understood that, if any prior art is referred to herein, such reference does not constitute an admission that the prior art forms a part of the common general knowledge in the art, in Australia or any other country.

### SUMMARY

Disclosed is a bandage for the treatment of a wound (e.g. a cut/lesion to the skin of an animal). The bandage may comprise a flexible layer comprising kinesiology tape. The flexible layer has outer and inner opposing faces extending between first and second opposing ends. The bandage may further comprise a pressure-sensitive adhesive disposed on at least a section of the inner face of the flexible layer. The bandage may further comprise an absorbent pad disposed on the inner face of the flexible layer and located to be positioned on the wound to promote healing of the wound. The flexible layer and the absorbent pad may be sized and disposed such that a portion of the pressure-sensitive adhesive retains the absorbent pad with respect to the flexible layer while a residual portion of the pressure-sensitive adhesive is free to adhere in use to itself or a surface such as a skin surface surrounding the wound to retain the bandage in position for treatment of the wound.

Advantageously, the bandage may be able to adhere to the surrounding wound area, allowing for difficult areas to be bandaged, such as knees, hocks, coronet bands and other flat surfaces. The pressure-sensitive adhesive ensures that the bandage does not slip after application. The flexible layer and in particular the composition of the flexible layer allows for the bandage to expand and contract as the swelling varies to inhibit problems such as bandage burns, pinched tendons and excessive swelling, as seen in the tightening and slipping of traditional bandages. Further, the combination of a flexible material and an adhesive allows for the bandage to adhere and contour to the region of the wound to inhibit debris from entering the bandage (e.g. there may be no gaps that facilitate the entrance of debris to the wound site). By combining the pad, adhesive and flexible material into a one-piece bandage, the time required to apply the bandage may be reduced.

In some forms, the bandage is substantially rectangular, square, round or oval in shape. In some forms, the bandage includes rounded corners.

In some forms, the bandage is scaled for large limbs or large wounds and is between 100 and 600 mm in length and between 100 and 400 mm in width.

In some forms, the flexible layer is configured to be able to be stretched between its ends such that the length of the bandage is able to be increased by up to 140% in use while the width of the bandage remains substantially constant. The continuous compression provided by the mono-directional elasticity of the bandage may allow for greater flow of venous and lymphatic fluids. This may also aid sensory pain receptors by applying a gentle lifting force to the skin directly over the treatment area.

In some forms, the pad is centrally located between the opposing ends of the flexible layer. In some forms, the pad is located towards the first end of the flexible layer to allow an expanded wrapping area of flexible layer.

In some forms, the absorbent pad comprises an alginate dressing configured to promote healing of the wound. The inclusion of an alginate dressing with the bandage advantageously results in the user not requiring the use of a separate dressing.

In some forms, the bandage further comprises a backing layer releasably adhered to the inner face of the flexible layer to inhibit the adhesive from drying or sticking to itself irremovably. In some forms, the pressure-sensitive adhesive is arranged to releasably adhere the backing layer to the inner face of the flexible layer.

In some forms, the backing layer comprises a wax coated paper material that is configured to cover the inner face of the flexible layer and the absorbent pad when adhered to the inner face of the flexible layer.

In some forms, the backing layer comprises a first backing layer segment and a second backing layer segment, the first segment overlapping the second segment such that a portion of an underside surface of the first segment is spaced from and thereby not adhered to the pressure-sensitive adhesive.

In some forms, the pad is scaled to cover large wounds while allowing sufficient residual area on the flexible layer for wrapping and holding the bandage in place. In some forms the absorbent pad is between 50 mm and 200 mm in length and between 50 mm and 300 mm in width.

In some forms, the pressure-sensitive adhesive comprises a plurality of spaced apart bands of pressure-sensitive adhesive that are disposed on the at least one section of the inner face of the flexible layer, wherein adjacent bands of pressure sensitive adhesive are separated by a gap that defines a region of the inner face of the flexible layer that does not include the pressure-sensitive adhesive. In some forms the space between the bands is smaller than or approximately the same width as the bands of adhesive.

In some forms, the plurality of strips of pressure-sensitive adhesive are each curved along a length of the strip to define a wave pattern.

In some forms, the pressure-sensitive adhesive is configured to adhere the bandage to an animal body.

Also disclosed herein is a method of treating an animal or human having a wound. The method may comprise locating a bandage on the wound, the bandage comprising a kinesiology tape having outer and inner opposing faces extending between first and second opposing ends, and an absorbent pad disposed on the inner face of the kinesiology tape and located to be positioned on the wound to promote healing of the wound; and pressing the bandage onto the wound such that a pressure-sensitive adhesive disposed on at least a section of the inner face of the kinesiology tape adheres to a surface surrounding the wound to retain the bandage in position for treatment of the wound.

Also disclosed herein is a method of manufacturing a bandage for the treatment of a wound. The method may comprise positioning a length of kinesiology tape having outer and inner opposing faces extending between first and second opposing ends; applying a pressure-sensitive adhesive to at least a section of the inner face of the kinesiology tape; positioning an absorbent pad such that the pad is retained by a portion of the adhesive with respect to the inner face of the kinesiology tape in a location that allows the pad to be positioned on the wound to promote healing of the wound, the kinesiology tape and the absorbent pad being sized such that a residual portion of the pressure-sensitive adhesive is free to adhere to a surface surrounding the wound to retain the bandage in position for treatment of the wound.

### BRIEF DESCRIPTION OF THE DRAWINGS

Embodiments will now be described by way of example only, with reference to the accompanying drawings in which

FIGS. 1a and 1b show a bandage of the prior art being applied (a) and removed (b) from an equine wound site;

FIGS. 2a and 2b show bottom (a) and top (b) views of an embodiment of the disclosed bandage;

FIGS. 3a and 3b show bottom views of the bandage of FIG. 2 covered (a) and partially covered (b) with a backing material;

FIGS. 4a and 4b show bottom views of the bandage of FIG. 2 partially covered with a backing material;

FIG. 5 shows an exploded view of the bottom of the bandage of FIG. 2;

FIG. 6 shows a perspective view of a user applying the bandage of FIG. 2 to the cannon bone of a horse;

FIG. 7 shows a perspective view of the bandage of FIG. 2 applied to the cannon bone of a horse;

FIG. 8 shows another perspective view of the bandage of FIG. 2 applied to the cannon bone of a horse;

FIG. 9 shows a perspective view of the bandage of FIG. 2 applied to the pastern and hoof of a horse;

FIG. 10 shows a perspective view of the bandage of FIG. 2 with the inclusion of Manuka honey to the pad;

FIG. 11 shows a perspective view of the bandage of FIG. 2 applied to the hock of a horse;

FIG. 12 shows a perspective view of the bandage of FIG. 2 applied to the neck of a horse;

FIG. 13 shows a perspective view of the bandage of FIG. 2 applied to the buttock of a horse; and

FIG. 14 shows bottom views of various embodiments of the bandage.

### DETAILED DESCRIPTION

In the following detailed description, reference is made to accompanying drawings which form a part of the detailed description. The illustrative embodiments described in the detailed description, depicted in the drawings and defined in the claims, are not intended to be limiting. Other embodiments may be utilised and other changes may be made without departing from the spirit or scope of the subject matter presented. It will be readily understood that the aspects of the present disclosure, as generally described herein and illustrated in the drawings can be arranged, substituted, combined, separated and designed in a wide variety of different configurations, all of which are contemplated in this disclosure.

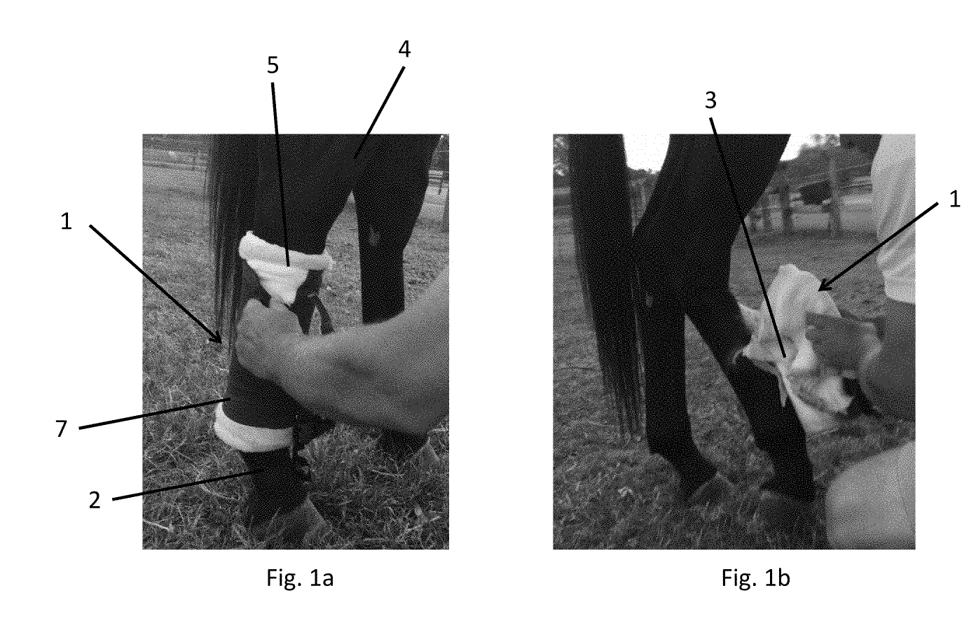

A layered bandage is commonly applied for the treatment of equine limb wounds. FIGS. 1a & b show a prior art equine bandage 1 being applied to (FIG. 1a) and removed from (FIG. 1b) the lower limb 2 of a horse 4. Once the wound is cleaned, a medicated dressing 3 is applied as a primary bandage to promote autolytic debridement. The medicated dressing is then be covered with a thick layer of padding 5 and secured to the wound with a further layer of material 7 that is wrapped around the equine limb. As previously described, this type of traditional equine bandage and bandaging method is problematic, expensive and time consuming.

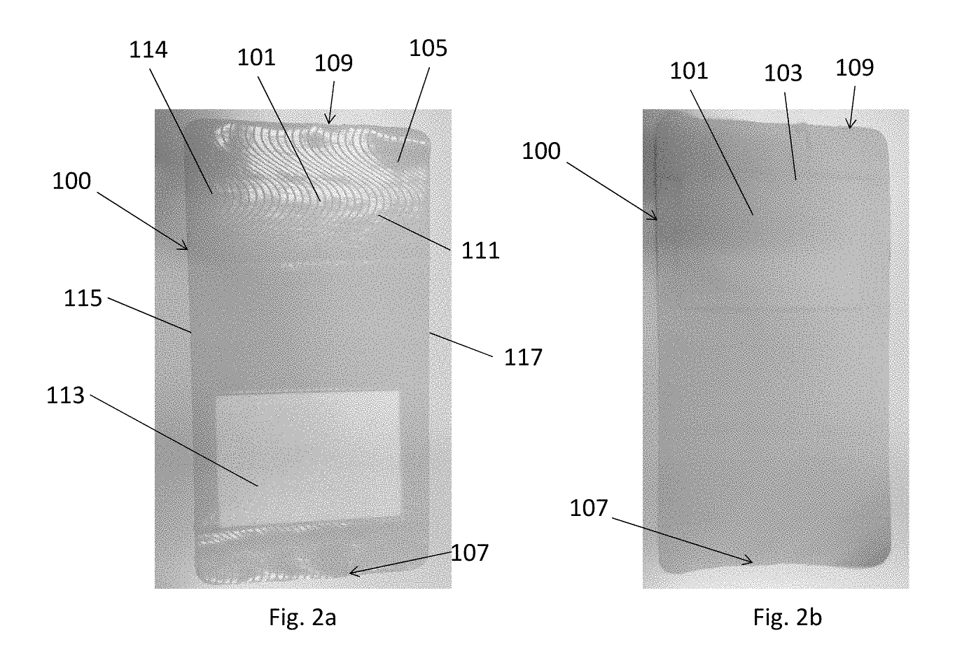

Disclosed herein is a bandage for the treatment of a wound. FIGS. 2a & 2b show a bottom (FIG. 2a) and top (FIG. 2b) view of the bandage 100 according to a first embodiment of the disclosure. The bandage 100 includes a flexible layer 101 that is formed from kinesiology tape. The kinesiology tape 101 has outer 103 and inner 105 opposing faces extending between first 107 and second 109 opposing ends.

Kinesiology tape is a latex free, non-medicated elastic tape that acts as a form of support. The elasticity of the tape allows it to be applied without significantly compromising the range of motion of the limb to which it is applied. Kinesiology tape is typically manufactured using cotton fibres that are thin and woven together to form an air permeable fabric. It differs from other elastic tapes in that it can be stretched up to 140% of its original length, applying a constant pulling force to the skin if applied when stretched. Moisture and air flow through the fabric minimizes skin irritation and allows the tape to be worn continuously over a long period of time. The elasticity of the fabric may lift the skin to relieve underlying pressure, provide greater drainage of the wound area and promote healing in the region of the wound.

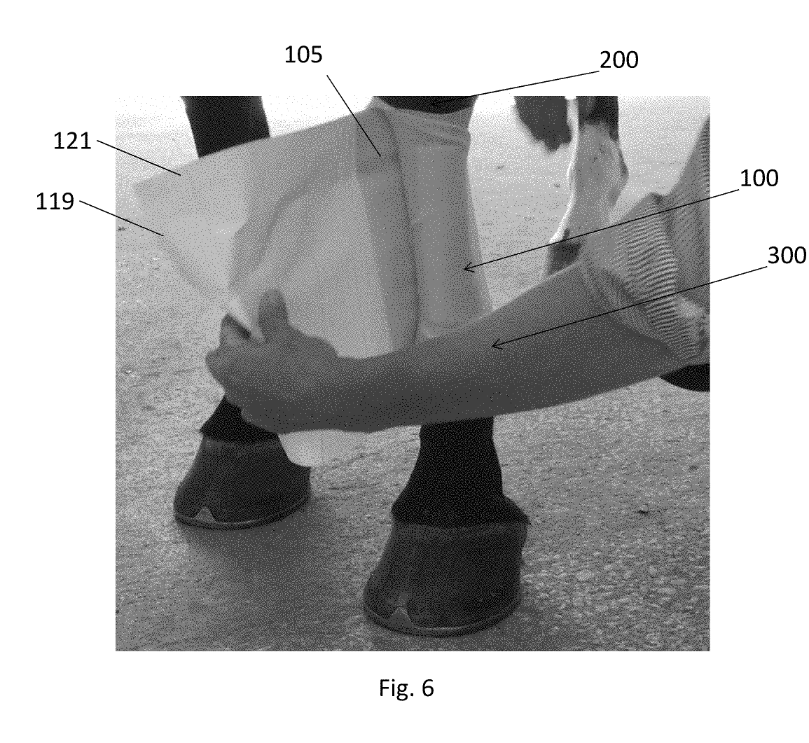

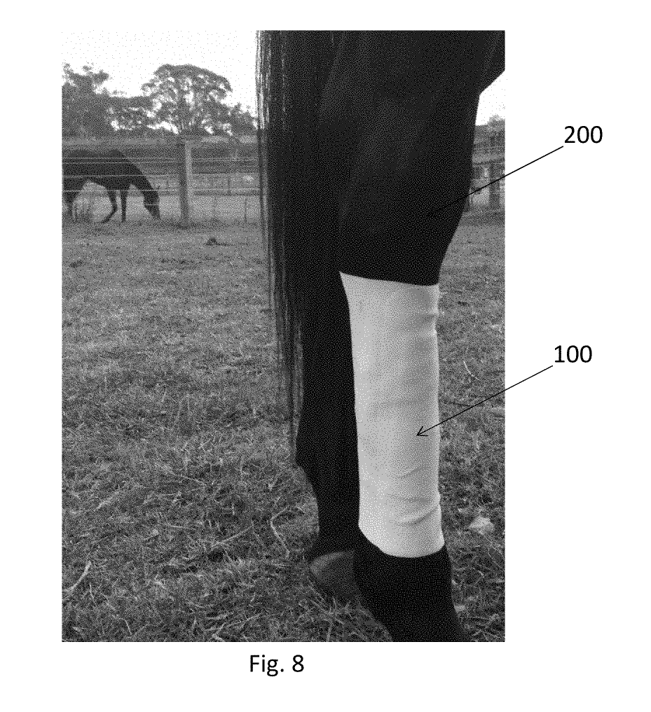

When the bandage is used for equine treatment, the elastic properties of the disclosed bandage advantageously allows the bandage to provide dynamic support to the injured area. The bandage may act as a second skin that protects joints and muscles. Due to its elastic properties, the bandage can allow for motion of the patients' body parts. Further, in at least one embodiment, the bandage may be worn during intense exercise, while showering and swimming. The elastic properties of the bandage also allow the bandage to be applied to uneven surfaces and joints that have been traditionally costly and difficult to bandage (e.g. the coronet band, neck, hindquarters, hock, knee and fetlock joints). The application of the bandage in the equine industry will be described in further detail with respect to FIGS. 6-13.

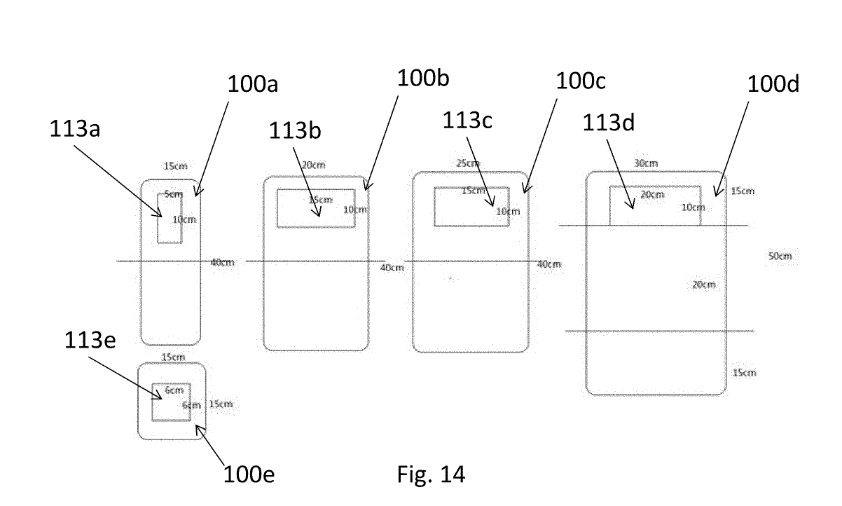

The bandage 100 is substantially rectangular in shape and includes rounded corners at each end 105, 107. The rounded corners facilitate application of the bandage and inhibit the corners from lifting away from the animal in use. In the detailed embodiment, the illustrated bandage is 400 mm in length (i.e. the distance between ends 105, 107 is 400 mm) and 250 mm in width (i.e. the distance between the sides 115, 117 is 250 mm). As will be evident to the skilled addressee, the size of the bandage can be adjusted to suit the application. Various embodiments will be described with reference to FIG. 14.

The bandage also includes a pressure-sensitive adhesive 111 disposed on at least a section of the inner face 105 of the kinesiology tape 101. In the detailed embodiment, the pressure-sensitive adhesive 111 is disposed between the first 107 and second 107 ends of the inner face 105 of the kinesiology tape 101. The bandage also includes an absorbent pad 113 disposed on the inner face 105 of the kinesiology tape 101. The pad 113 is disposed with respect to the inner face 105 such that it can be positioned on the wound of the animal to promote healing of the wound. In the detailed embodiment, the pad 113 is 100 mm in length and 150 mm in width. Further, the pad 113 is positioned towards the first end 107 of bandage (i.e. it is offset towards the first end and not centrally located between the first and second ends). As will be evident to the skilled addressee, the size and location of the pad can be adjusted to suit the application. The pad size will typically be smaller than the size of the kinesiology tape. Various embodiments will be described with reference to FIG. 14.

In one embodiment, the pad is in the form of an alginate dressing. The alginate dressing is a flexible, highly absorbent, biodegradable material derived from seaweed. Alginate dressings are useful to cleanse a wide variety of secreting lesions. The high absorption is achieved via strong hydrophilic gel formation. The pad may limit wound secretions and minimises bacterial contamination. Advantageously, alginate fibres trapped in a wound are biodegraded. Alginate dressings can maintain a moist microenvironment that promotes healing and the formation of granulation tissue. Alginates can be rinsed away with saline irrigation, so removal of the dressing does not interfere with healing granulation tissue. Alginate pads are flexible and thus can be deformed to suit the shape of the wound and limb to which it is being applied. Further, alginate pads can absorb up to 11 times liquid of its own weight to keep the wound clean.

The kinesiology tape 101 and the absorbent pad 113 are sized and positioned such that a portion (not shown as this portion is behind the pad 113 in FIG. 2) of the pressure-sensitive adhesive 111 retains the absorbent pad with respect to the kinesiology tape while a residual portion 114 of the pressure-sensitive adhesive is free to adhere in use to a surface surrounding the wound to retain the bandage in position for treatment of the wound. To manufacture the bandage, the pressure-sensitive adhesive maybe applied to the inner surface 105 of the bandage. The pad 113 can then be located on the pressure-sensitive adhesive 111 and inner surface 105 and then pressed (e.g. forced) towards the inner surface 105 to thereby adhere the pad 113 to the inner surface kinesiology tape 101.

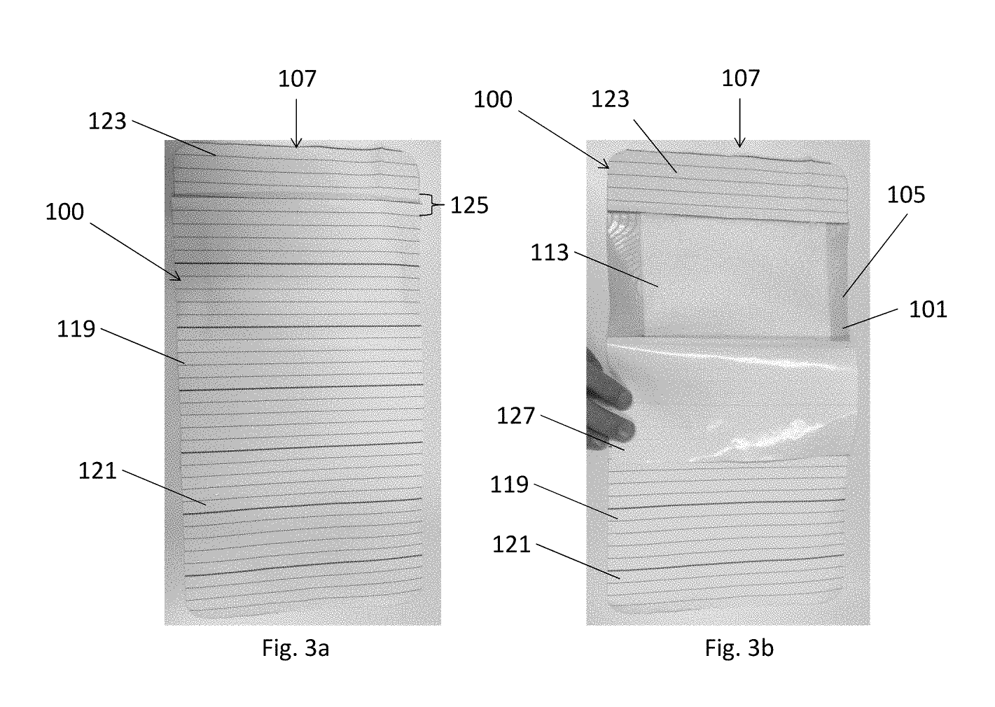

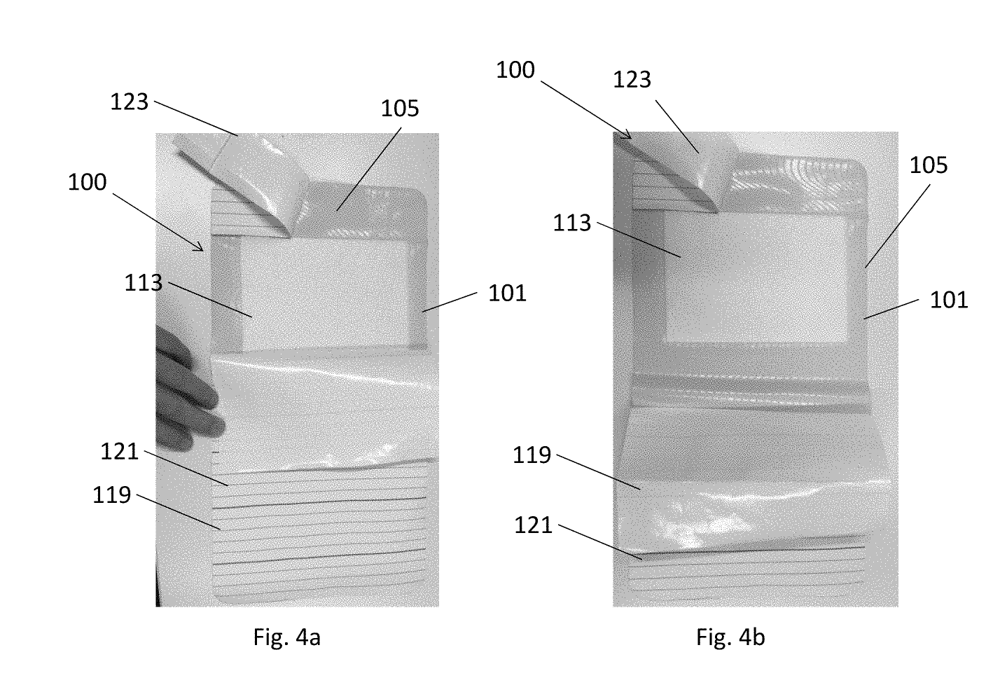

As shown in FIGS. 3a-b and FIGS. 4a-c, the bandage includes a backing layer, in the form of a wax coated paper material 119, releasably adhered to the inner face 105 of the kinesiology tape 101. The wax coated paper material 119 inhibits the adhesive 111 from drying. The paper material 119 is configured to cover the inner face 105 of the kinesiology tape 101 and the absorbent pad 113 when adhered to the inner face 105 of the kinesiology tape 101. In addition to inhibiting the adhesive from drying, by covering the pad (i.e. adhered to the inner surface on all sides of the pad) the backing material may be able to keep the pad clean until the paper material is removed to reveal the pad and adhesive for use.

The wax coated paper material 119 includes a first backing layer segment 121 and a second backing layer segment 123. The first segment 121 overlaps the second segment 123 such that a portion 125 (see FIG. 3a) of an underside surface 127 (see FIG. 3b) of the first segment 121 is spaced from and thereby not adhered to the pressure-sensitive adhesive 111. A user is able to grip this portion 127 of the first segment 121 and lift the first segment 121 of the wax coated paper material 119 away from the inner face 105 of the bandage 100 (see FIGS. 3b and 4b). To remove the second segment 123 from the inner face 105 of the bandage 100, the user is able to lift a corner of the second segment 123 and pull it away from the inner face 105 (see FIGS. 4a & 4b). Removing the first 121 and second 123 segments of the wax coated paper material 119 exposes the pad 113 and the adhesive 111 on the inner face 105 such that a user is then able to apply (e.g. stick, adhere) the bandage 100 to the surface of the animal. The pad 113 is positioned over the wound (e.g. a lesion) and the user is then able to apply pressure to the outer surface 103 of the bandage to stick the bandage to the animal.

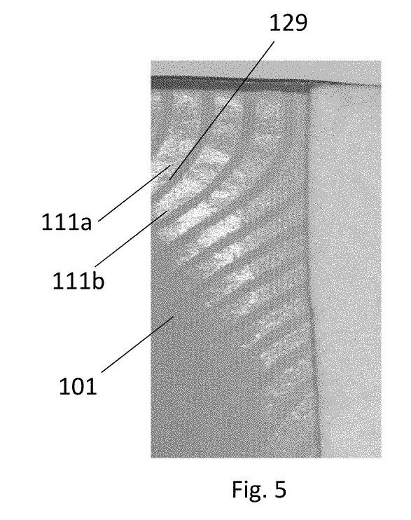

As shown in FIG. 5, the pressure-sensitive adhesive 111 comprises a plurality of bands 111a, 111b of pressure-sensitive adhesive that are disposed on the inner face of the kinesiology tape 101. Adjacent bands 111a, 111b of pressure sensitive adhesive are separated by a gap 129 that defines a region of the inner face of the kinesiology tape 101 that does not include the pressure-sensitive adhesive. In the illustrated form the bands are separated by a gap that is smaller than the width of the bands. Advantageously, this allows for moisture (e.g. sweat) to pass through the bandage. In the detailed embodiment, the plurality of strips 111a, 111b of pressure-sensitive adhesive are each curved along their length and define a wave pattern (e.g. a sinusoidal wave pattern).

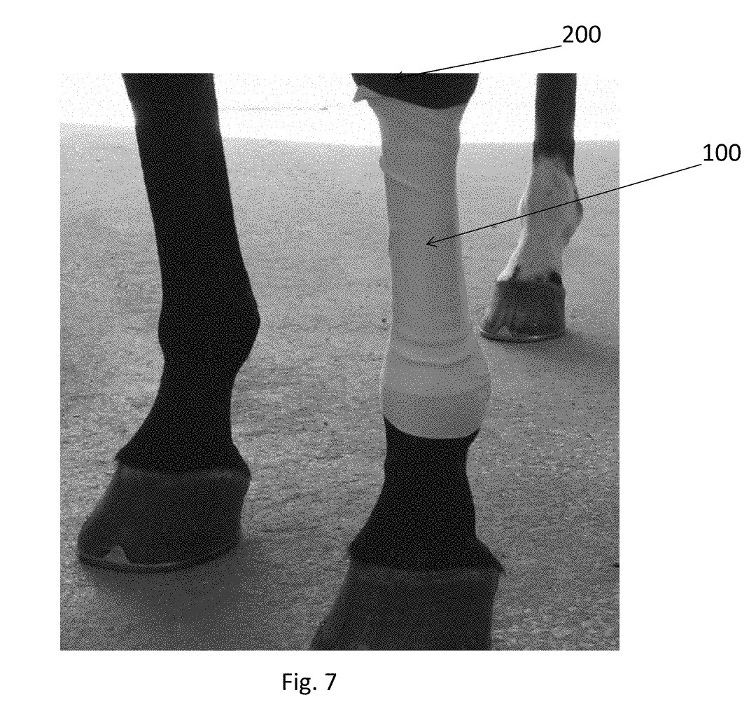

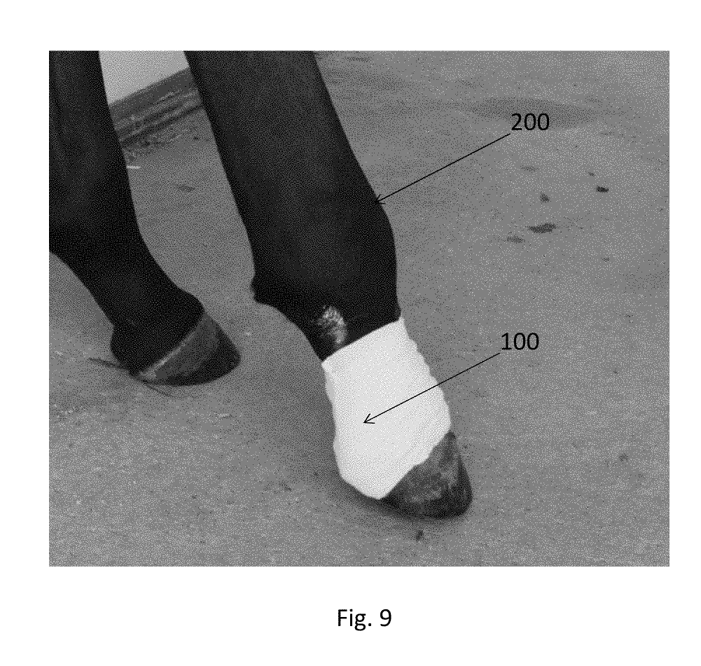

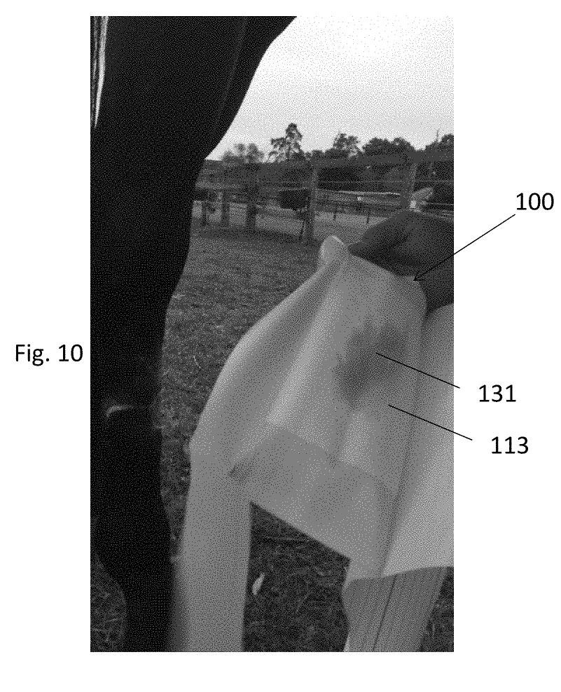

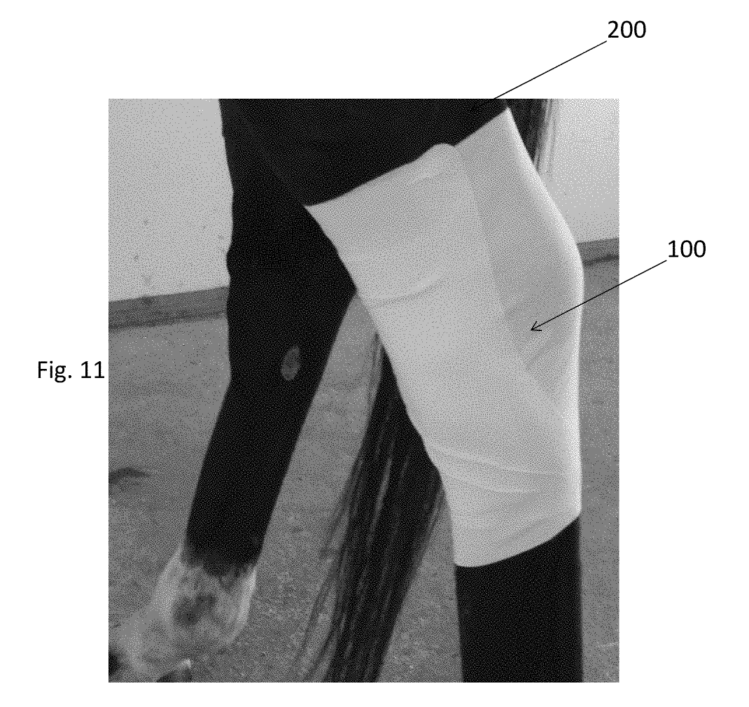

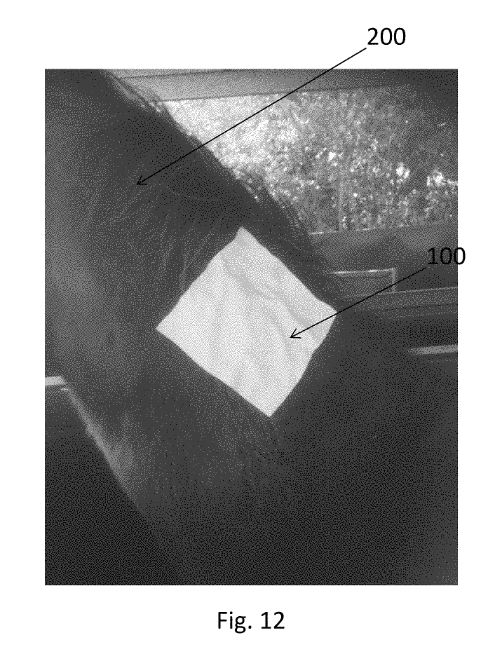

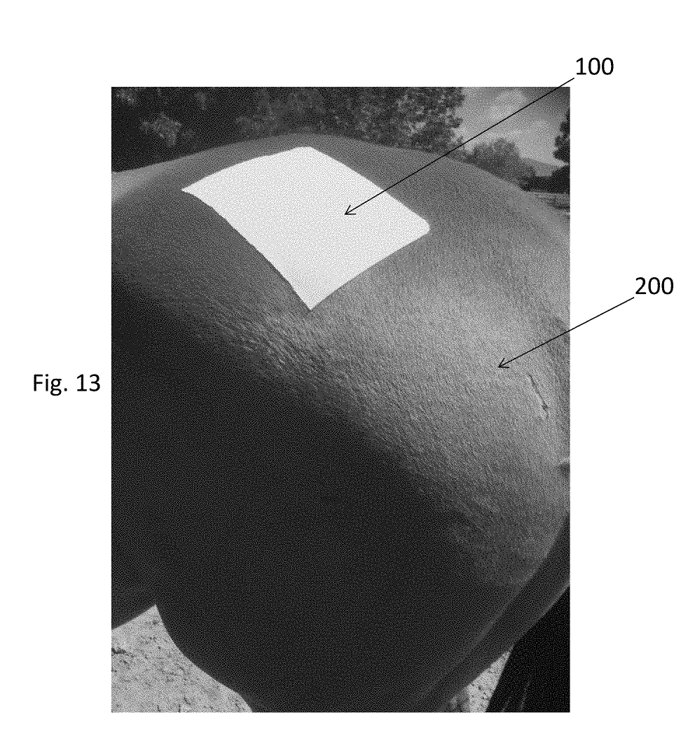

FIGS. 6-13 show the bandage applied to various equine anatomy. FIGS. 6-8 show the bandage 100 applied to the cannon bone of a horse 200. FIG. 6 shows a person 300 wrapping the bandage 100 around the cannon bone of the horse 200. The wax coated paper material 119 is still partially adhered to the inner surface 105 of the bandage 100. After removing second segment of the backing paper material 119 to expose a portion of the inner face 105 and adhesive, the user applies the bandage 100 such that the pad (not shown) is positioned over the wound and then applies pressure to the bandage to adhere the bandage to the skin of the horse. The user then holds the first segment 121 of the wax coated paper material 119 and wraps the bandage around the cannon bone. The bandage adheres to both the cannon bone and to itself (i.e. as the bandage folds onto itself) as it is continuously wrapped around the cannon bone. FIGS. 7 & 8 show the bandage after it has been applied to the cannon bone. FIG. 9 shows the bandage applied to the pastern and hoof. FIG. 10 shows an embodiment of the bandage whereby Manuka honey has been applied to the pad prior to use. FIG. 11 shows the bandage 100 applied to the hock. This area is traditionally very difficult to bandage, as a result of the uneven surface and location of the joint. FIGS. 12 & 13 show the bandage applied to the neck (FIG. 12) and buttock (FIG. 13) of a horse.

FIG. 14 shows various embodiments of the bandage 100a-100e having different sizes. The length and width of the bandage can be varied to suit the application. In the embodiments shown in FIG. 14, the bandage varies from is between 150 mm (100e) and 500 mm in length (100d) and between 150 mm (100e) and 300 mm (100d) in width. The length and width of the pad 113a-e can also be varied to suit the application. In the embodiments shown in FIG. 14, the pad 113a-e varies from is between 60 mm (113e) and 100 mm in length (113d) and between 50 mm (113a) and 200 mm (113d) in width. The pad can also be disposed towards an end of the bandage (e.g. 113a-d) or centrally located (113e) with respect to the pad. As will be evident to the skilled addressee, the pad can be positioned at various positions with respect to the bandage in dependence on the application of the bandage.

Variations and modifications may be made to the parts previously described without departing from the spirit or ambit of the disclosure.

For example the pad can be impregnated with antibiotics (both natural and synthetic), antibacterial, antiviral, anti-fungal and anti-inflammatory substances. This includes but is not limited to colloidal silver, Manuka honey, aloe vera, peroxide, Iodine etc. Healing dressing such as hydrocolloid/hydrogel can also be utilised. In another embodiment, the pad may be impregnated with a pain relief material (e.g. gel).

In some forms, the bandage combines cooling with compression to speed up recovery time. The bandage may draw heat from the injured area through evaporation to reduce pain and swelling. As such, the bandage may not be pre-refrigerated prior to being applied. As the bandage does not restrict the range of movement around the injured area, it may be ideal for treating inflammation and bruising of muscles, tendons, joints and ligaments.

In some forms, the bandage may be configured to heat the wound region (e.g. to release the tension in a tight muscle). The bandage may contain a soft, flexible backing that adheres to any injured area, and can be removed easily. In this embodiment, the bandage may be designed to warm the wounded region gradually and reach maximum heat in approximately 30 minutes. The bandage may be configured to deliver a sustained temperature of above 40 degrees for approximately 12-15 hours.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A bandage for the treatment of a wound, the bandage comprising:
a flexible layer comprising kinesiology tape, the flexible layer having outer and inner opposing faces extending between first and second opposing ends;
a pressure-sensitive adhesive disposed on at least a section of the inner face of the flexible layer;
an absorbent pad disposed on the inner face of the flexible layer and located to be positioned on the wound to promote healing of the wound;
the flexible layer and the absorbent pad being sized and disposed such that a portion of the pressure-sensitive adhesive retains the absorbent pad with respect to the flexible layer while a residual portion of the pressure-sensitive adhesive is free to adhere in use to itself or a surface surrounding the wound to retain the bandage in position for treatment of the wound.

2. A bandage in accordance with claim 1, wherein the bandage is substantially rectangular in shape.

3. A bandage in accordance with claim 1, wherein the bandage is substantially square in shape.

4. A bandage in accordance with any one of the preceding claims, wherein the bandage is between 100 and 600 mm in length and between 100 and 400 mm in width.

5. A bandage according to claim 4, wherein the flexible layer is configured to be able to stretch between its ends such that the length of the bandage is able to be increased by up to 140% in use while the width of the bandage remains substantially constant.

6. A bandage in accordance with any one of the preceding claims, wherein the pad is centrally located between the opposing ends of the flexible layer.

7. A bandage in accordance with any one of claims 1 to 6, wherein the pad is located proximal the first end of the flexible layer.

8. A bandage according to any one of the preceding claims, wherein the pad comprises an alginate dressing configured to promote healing of the wound.

9. A bandage according to any preceding claim, further comprising a backing layer releasably adhered to the inner face of the flexible layer to inhibit the adhesive from drying.

10. A bandage according to claim 9, wherein the pressure-sensitive adhesive is arranged to releasably adhere the backing layer to the inner face of the flexible layer.

11. A bandage according to claim 10 wherein the backing layer comprises a wax coated paper material that is configured to cover the inner face of the flexible layer and the absorbent pad when adhered to the inner face of the flexible layer.

12. A bandage according to claim 11, wherein the backing layer comprises a first backing layer segment and a second backing layer segment, the first segment overlapping the second segment such that a portion of an underside surface of the first segment is spaced from and thereby not adhered to the pressure-sensitive adhesive.

13. A bandage in according to any one of the preceding claims, wherein the pad is between 50 mm and 200 mm in length and between 50 mm and 300 mm in width.

14. A bandage according to any one of the preceding claims, wherein the pressure-sensitive adhesive comprises a plurality of bands of pressure-sensitive adhesive that are disposed on the at least one section of the inner face of the flexible layer, wherein adjacent bands of pressure sensitive adhesive are separated by a gap that defines a region of the inner face of the flexible layer that does not include the pressure-sensitive adhesive.

15. A bandage according to claim 14, wherein the plurality of bands of pressure-sensitive adhesive are each curved along a length of the strip to define a wave pattern.

16. A bandage according to any one of the preceding claims wherein the pressure-sensitive adhesive is configured to adhere the bandage to an animal body.

17. A method of treating an animal or human having a wound, the method comprising:
taking a bandage comprising kinesiology tape having outer and inner opposing faces extending between first and second opposing ends, and an absorbent pad disposed on the inner face of the kinesiology tape and located to be positioned on the wound to promote healing of the wound;
locating the absorbent pad on the wound, and
pressing the bandage onto a surface surrounding the wound such that a pressure-sensitive adhesive disposed on at least a section of the inner face of the kinesiology tape adheres to the surface surrounding the wound to retain the bandage in position for treatment of the wound.

18. A method according to claim 17, wherein the bandage is as otherwise defined in any one of claims 1 to 16.

19. A method of manufacturing a bandage for the treatment of a wound, the method comprising:
positioning a length of kinesiology tape having outer and inner opposing faces extending between first and second opposing ends;
applying a pressure-sensitive adhesive to at least a section of the inner face of the kinesiology tape;
positioning an absorbent pad such that the pad is retained by a portion of the adhesive with respect to the inner face of the kinesiology tape in a location that allows the pad to be positioned on the wound to promote healing of the wound, the kinesiology tape and the absorbent pad being sized such that a residual portion of the pressure-sensitive adhesive is free to adhere to a surface surrounding the wound to retain the bandage in position for treatment of the wound.

20. A method according to claim 19, wherein the bandage is as otherwise defined in any one of claims 1 to 16.
